(19) United States
(12) Patent Application Publication
(10) Pub. No.: US 2022/0079885 A1
(43) Pub. Date: Mar. 17, 2022

(54) EXPANDABLE MICROCAPSULE

(71) Applicant: TZU PIN WANG, TAICHUNG CITY (TW)

(72) Inventor: TZU PIN WANG, TAICHUNG CITY (TW)

(21) Appl. No.: 17/465,126

(22) Filed: Sep. 2, 2021

(30) Foreign Application Priority Data
Sep. 14, 2020 (TW) 109131469

(51) Int. Cl. A61K 9/50; A61K 9/46

(52) U.S. Cl. CPC A61K 9/50; A61K 9/0007

(57) ABSTRACT

An expandable microcapsule, which includes a core layer consisting of 300 parts by weight of foaming agent, 250 parts by weight of polymer water-absorbing material and 60 parts by weight of core layer lubricant, wherein the foaming agent has characteristics of reacting with water to generate gas; and a coating layer covering the surface of the core layer to form a granular structure, wherein the coating layer is consisting of 10% to 40% by weight of edible water-absorbent colloid, 5% to 40% by weight of functional health food additives, 2% to 30% by weight of emulsifier, 4% to 15% by weight of coating layer lubricant and 5% to 40% by weight of film coating composition, and wherein the film coating composition has characteristics of resistance to gastric acid decomposition and being decomposed only in the intestinal tract.

### FIELD OF THE INVENTION

The present disclosure is an expandable microcapsule that can physically expand in the human stomach after consumption to increase satiety.

### BACKGROUND OF THE INVENTION

“Obesity” usually refers to abnormal or excessive fat accumulation in the human body. According to the results of nutrition and health surveys, obesity can increase the burden of physical functions, major risk factors for non-communicable diseases, and lead to the occurrence of many chronic diseases. For example, it is easy to cause diseases such as arteriosclerosis, heart disease, stroke, cardiovascular disease, high blood pressure, hyperlipidemia, diabetes, malignant tumor, fatty liver, metabolic syndrome and so on.

Although drugs such as metabolic stimulants, appetite suppressants, and starch blockers can achieve weight loss in a short period of time, long-term use of drugs is bound to cause many adverse side effects to the human body. For example, the current legal weight-loss drugs, Roche Fresh® (Orlistat®), is an inhibitor of gastrointestinal pancreatic lipolytic enzymes, which can prevent part of the fat in food from being broken down and absorbed in the gastrointestinal tract, thereby reducing the calories of fat from the food to achieve the effect of weight loss. However, Orlistat® has lots of side effects, including interference with the absorption of fat-soluble vitamins, oily stools, bloating, and possibly gallstones and liver and kidney damage, so it is not suitable for long-term use. There are also weight loss drugs with phentermine-topiramate compound and lorcaserin as the main ingredients. The mechanism of action is to increase satiety and suppress appetite to achieve weight loss, but there will be dizziness, nausea or insomnia, constipation and other side effects. In addition, these two weight loss drugs still have doubts about the risk of cardiovascular disease, and they are not suitable for patients with cardiovascular disease.

### SUMMARY OF THE INVENTION

The main purpose of the present disclosure is to provide an expandable microcapsule, which has the characteristics of being able to physically expand in the human stomach after consumption, thereby increasing satiety and suppressing appetite, so as to achieve the purpose of weight loss, and not to cause any adverse side effects of drugs to the human body.

To achieve the foregoing objective, the present disclosure provides an expandable microcapsule, which includes:

a core layer consisting of 300 parts by weight of foaming agent, 250 parts by weight of polymer water-absorbing material and 60 parts by weight of core layer lubricant, wherein the foaming agent has characteristics of reacting with water to generate gas; and

a coating layer covering the surface of the core layer to form a granular structure, wherein the coating layer is consisting of 10% to 40% by weight of edible water-absorbent colloid, 5% to 40% by weight of functional health food additives, 2% to 30% by weight of emulsifier, 4% to 15% by weight of coating layer lubricant and 5% to 40% by weight of film coating composition, and wherein the film coating composition has characteristics of resistance to gastric acid decomposition and being decomposed only in the intestinal tract.

Preferably, the eater not only directly eat a predetermined amount of the expandable microcapsules to make them physically expand in the human stomach to increase satiety and suppress appetite, but also tablets which is made according to needs; or, it is also possible to fill a predetermined amount of the expandable microcapsules into a hollow capsule composed of two symmetrical half capsules, so as to facilitate the consumption of the eater.

### BRIEF DESCRIPTION OF THE DRAWINGS

FIG. 1 is a schematic diagram of the composition structure of the expandable microcapsule of the present disclosure.

FIG 2 is a schematic diagram of the physical expansion of the expandable microcapsule of the present disclosure.

FIG. 3 is a schematic diagram of the composition structure of the tablet of the expandable microcapsule of the present disclosure.

FIG. 4 is a schematic diagram of the composition structure of another embodiment of the expandable microcapsule of the present disclosure.

### DETAILED DESCRIPTION OF THE PREFERRED EMBODIMENTS

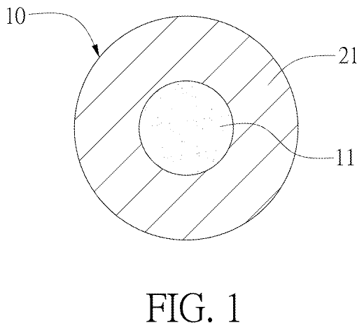

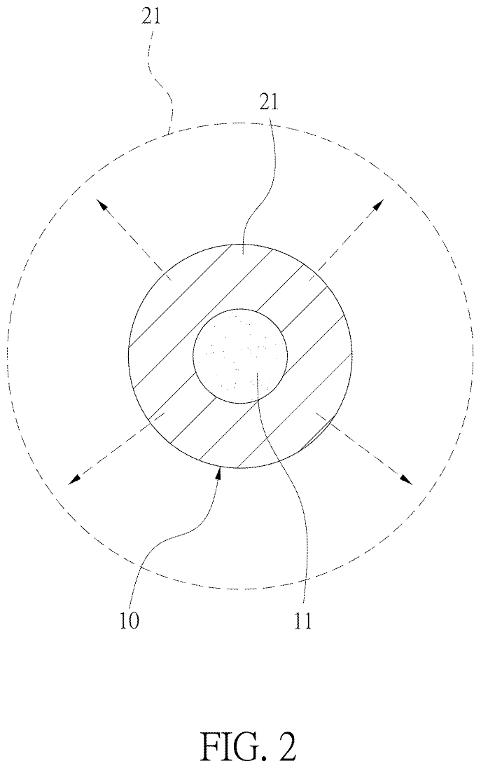

First, please refer to FIG. 1 and 2, which is an expandable microcapsule 10 provided by the present disclosure, which is mainly composed of a core layer 11 and a coating layer 21 covering the surface of the core layer 11, wherein:

the core layer 11 consists of 300 parts by weight of foaming agent, 250 parts by weight of polymer water-absorbing material and 60 parts by weight of core layer lubricant, and the foaming agent has the characteristics of reacting with water to generate gas; and

the coating layer 21 covering the surface of the core layer 11 to form a granular structure, the coating layer 21 is consisting of 10% to 40% by weight of edible water-absorbent colloid, 5% to 40% by weight of functional health food additives, 2% to 30% by weight of emulsifier, 4% to 15% by weight of coating layer lubricant and 5% to 40% by weight of film coating composition, and wherein the film coating composition has the characteristics of resistance to gastric acid decomposition and being decomposed only in the intestinal tract.

In actual production, the foaming agent of the present disclosure is composed of 100 to 300 parts by weight of acidic compounds and 130 to 410 parts by weight of basic compounds, and the foaming agent is dried for 2 to 4 hours at 40° C. to 80° C. before use, and passed through a 40 to 100 mesh sieve. The acidic compound and the basic compound are preferably: 200 parts by weight of the acidic compounds and 270 parts by weight of the basic compounds; or 240 parts by weight of the acidic compounds and 400 parts by weight of the basic compounds; or 160 parts by weight of the acidic compounds and 140 parts by weight of the basic compounds.

Preferably, the above-mentioned basic compound may be one of sodium carbonate, sodium bicarbonate, potassium carbonate, potassium bicarbonate, calcium carbonate, sodium glycine carbonate, potassium glycine carbonate, or any combination thereof. In addition, all edible alkali metal or alkaline earth metal carbonates, percarbonates, bicarbonates and their mixed carbonates can be used as basic compounds that can generate carbon dioxide gas. Among the basic compounds, at least one of sodium carbonate, sodium bicarbonate, potassium carbonate, and potassium bicarbonate is preferred. In addition, considering that taking sodium-containing foaming agents multiple times a day will cause adverse consequences for eaters who are not suitable to eat more sodium, it can be taken into account that using less sodium carbonate and sodium bicarbonate but using potassium bicarbonate, potassium carbonate, calcium carbonate, zinc carbonate and other non-sodium or low-sodium carbon dioxide sources used as a substitute.

Preferably, the polymer water-absorbing material of the present disclosure is sodium polyacrylate, and the polymer water-absorbing material is superfinely pulverized to a size of 300 mesh or more, and dried at 40° C. to 80° C. for 2 to 5 hours.

The core layer lubricant of the present disclosure mainly consists of 20 to 60 parts by weight of internal lubricant and 2 to 6 parts by weight of external lubricant. Preferably, the internal lubricant is polyethylene glycol 4000 or polyethylene glycol 6000, and the external lubricant is magnesium stearate, talc, or L-leucine or DL-leucine, or any combination thereof. The internal lubricant and the external lubricant are preferably: 40 parts by weight of the internal lubricant and 4 parts by weight of the external lubricant, or 25 parts by weight of the internal lubricant and 5 parts by weight of the external lubricant, or 57 parts by weight of the internal lubricant and 3 parts by weight of the external lubricant.

The core layer 11 of the present disclosure is prepared by mixing the foaming agent, the polymer water-absorbing material, and the internal lubricant according to the above formula ratio, sieving (according to product requirements), mixing homogeneously, and adding 240 parts by weight of a soft material made of anhydrous ethanol. After granulation by sieving (according to product requirements), drying at 40° C. to 80° C. to make the water content of the mixture particles below 3 then sieving and finishing (according to product requirements), and then adding the external lubricant, mixing homogeneously, and the granular core layer 11 of the present disclosure can be obtained.

Next, each composition of the coating layer 21 of the present disclosure is described as follows, wherein:

the coating layer is consisting of 10% to 40% by weight of edible water-absorbent colloid, 5% to 40% by weight of functional health food additives, 2% to 30% by weight of emulsifier, 4% to 15% by weight of coating layer lubricant and 5% to 40% by weight of film coating composition. The edible water-absorbent colloid in the coating layer 21 includes naturally occurring materials, such as plant secretions, seed gums and seaweed extracts, or can also be chemically modified materials, such as modified cellulose, starch or natural rubber derivatives. In a feasible embodiment, the edible water-absorbing colloid is composed of pectin, konjac powder, arabic gum, carrageenan, alginate, agar, guargum, xanthan gum, locust bean gum, gelatin, gellan gum, galactomannan, tragacanth gum, karaya gum, curdlan gum, chitosan, xyloglucan, β-glucan, furcellaran, Ghattigum, tamarin, bacterial gum, propylene glycol alginate, carboxymethyl locust bean gum, low methoxy pectin, mixture of emulsified calcium and pectin, modified microcrystalline cellulose, modified carboxymethyl cellulose (CMC), modified methyl cellulose (MC), modified hydroxypropyl methyl cellulose (HPMC), modified hydroxypropyl cellulose (HPC), or any combination thereof.

The functional health food additives in the coating layer 21 include, but are not limited to, cellulose, probiotics or vitamins that are beneficial to the human body, as well as all that can help digestion, improve metabolism, promote fat burning, help smooth bowel movements, inhibit sugars and fat absorption and other beneficial additives to the human body.

The film coating composition in the coating layer 21 is designed to remain intact and not digested in the acidic environment of the stomach and can absorb the water in the stomach, but it can be dissolved in the more alkaline environment of the intestine. In some embodiments, the film-forming agent includes shellac methyl cellulose, ethyl cellulose, propyl methyl cellulose, hydroxypropyl methyl cellulose phthalate, pectin, seaweed gum, sodium alginate, hydroxypropyl cellulose, hydroxypropyl methyl cellulose, hydroxyethyl cellulose, polyvinylpyrrolidone, polyethylene glycol aminoalkyl methacrylate copolymer, maltodextrin and polydextrose, or any combination thereof. Preferably, the film-forming agent with stomach insolubility is sodium alginate. In addition, based on 100% by weight of the film coating composition, the film coating composition includes 1% to 25% of the film-forming agent, 0.5% to 15% of the anti-sticking agent, and 60% to 95% of the solvent. In addition, an emulsifier can be further added to the coating layer 21, the mass ratio of the edible water-absorbant colloid to the emulsifier can be 7:1, and the emulsifier can be particularly selected from lecithin, fatty esters of glycerol, sucrose or sorbitol, in order to be used in the environment where there is a high content of protein or oil. Furthermore, according to production needs, add a coating layer lubricant that is convenient for granulation, the coating layer lubricant is composed of one of polyethylene glycol 4000, polyethylene glycol 6000, sodium lauryl sulfate, magnesium lauryl sulfate, L-leucine, sodium benzoate, sodium oleate, sodium chloride, sodium acetate, boric acid, magnesium stearate, talc, micronized silica gel, sucrose fatty acid ester, sodium stearyl fumarate, L-leucine and DL-leucine, or any combination thereof.

Preferably, the method for coating the surface of the core layer with the coating layer 21 of the present disclosure can be made into a granular structure by conventional pharmacological techniques. Conventional pharmacological techniques include, for example, one or a combination of the following methods: (1) dry mixing, (2) direct compression, (3) grinding, (4) dry or non-aqueous granulation, (5) wet granulation, (6) melting, or (7) extrusion/spheronization. Other methods such as commonly used solvent evaporation methods (e.g. spray drying, stratification or fluidized bed granulation, rotating disk, three-phase emulsification technology, etc.). Suitable solvents for solvent evaporation include alcohols (e.g. methanol, ethanol, n-propanol, isopropanol and butanol), ketones (e.g. acetone, methyl ethyl ketone and methyl isobutyl ketone), esters (e.g. ethyl acetate and propyl acetate) and various other solvents such as isopropyl ether, acetonitrile, dichloromethane, chloroform, hexane, toluene, tetrahydrofuran, cyclic ethers, and 1,1,1-trichloroethane; low volatility solvent can also be used, such as dimethyl acetamide or dimethyl sulfide. Mixtures of solvents can also be used, such as 20% ethanol, 80% acetone, and their mixtures with water.

The expandable microcapsule 10 of the present disclosure made of the above-mentioned components can remove water generated by the inevitable acid-base reaction in time by adding a polymer water-absorbing material to the core layer 11, so that the particle surface of the foaming agent in the core layer can be isolated, and kept in a dry state to prevent the acid-base reaction, thereby solving the problem that the foaming agent is unstable due to easily absorbing moisture and flatulence. Also, under actual action, it can be ensured that the effect of the foaming agent at a fast foaming speed after absorbing water. On the other hand, absorbing water through the polymer water-absorbing material is beneficial to stabilize the colloidal shape of the coating layer and have an excellent molding effect.

When the expandable microcapsule 10 of the present disclosure enters the human stomach after being eaten, the edible water-absorbent colloid of the coating layer 21 will be softened after absorbing water, and the core layer 11 will also swell due to the absorption of water. Since the foaming agent in the core layer 11 reacts with water to generate carbon dioxide, the coating layer 21 covering the core layer 11 can be blown by the continuous carbon dioxide generated by the foaming agent and rapidly expand and become larger (as shown in FIG. 2). In this way, the effect of physical swelling of the expandable microcapsule can be achieved, and by this, the satiety of the eater can be greatly increased, thereby suppressing the appetite. On the other hand, the film coating composition with a specific composition in the coating layer 21 will not decompose and collapse in the acidic environment of the stomach, so it can maintain its swollen body in the stomach. However, when it enters the more basic environment of the intestine, it will begin to disintegrate. Therefore, when an eater eats the expandable microcapsule 10 of the present disclosure, the releasing of the functional health food additives contained in the coating layer 21 can be delayed until reaching the intestinal tract without being decomposed and destroyed by gastric acid, thereby increasing the concentration absorbed by the human body to enhance its efficacy. The expandable microcapsule provided by the present disclosure have the characteristics of physical expansion in the human stomach, and thereby increasing satiety and suppress appetite, so as to achieve the purpose of weight loss. The expandable microcapsule does not contain any pharmaceutical ingredients, and thus to ensure that it will not cause any adverse side effects to the human body.

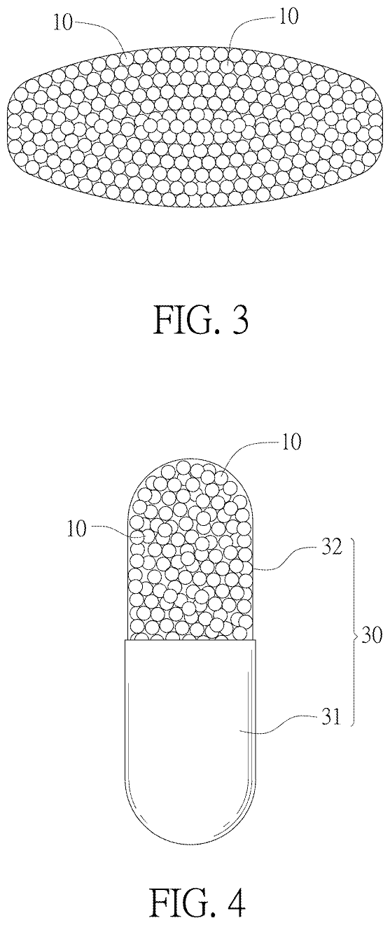

In addition, in practical applications of the expandable microcapsules provided by the present disclosure, the eater not only directly eat a predetermined amount of the expandable microcapsules 10 to make them physically expand in the human stomach to increase satiety and suppress appetite, but also according to needs tablets as shown in FIG. 3 made by the above-mentioned conventional pharmacological technologies with adding a suitable binder and organic solvent. The binder is used to impart the effect of binding a plurality of expandable microcapsules 10 to each other, and includes, for example, alginic acid and its salts; cellulose derivatives such as carboxymethyl cellulose, methyl cellulose, and hydroxypropyl methyl cellulose, hydroxyethyl cellulose, hydroxypropyl cellulose, ethyl cellulose (e.g., microcrystalline cellulose); microcrystalline dextrose; amylose; magnesium aluminum silicate; polysaccharide acid; bentonite; gelatin; polyvinylpyrrolidone/vinyl acetate copolymer; crospovidone; povidone; starch; pregelatinized starch, tragacanth gum, dextrin; sugars such as sucrose, glucose, dextrose, molasses, mannitol, sorbitol, xylitol and lactose; natural or synthetic gums, such as acacia, tragacanth gum, ghatti gum, povidone, isabella husk mucus; polyvinyl pyrrolidone, polymethacrylate, arabinogalactan, polyethylene glycol, propylene glycol, poly(ethylene oxide), wax, sodium alginate; also include (for example) one or any combination of agar, alginic acid, carbomer, carrageenan, guar gum, cellulose acetate phthalate, stearic acid, ceratonia and povidone. The organic solvent can be selected from ethanol, isopropyl alcohol, tetrahydrofuran, isopropyl ether, acetone, methyl ethyl ketone, methylene chloride or a mixture of these solvents.

In another embodiment of the present disclosure shown in FIG. 4, a predetermined number of the expandable microcapsules 10 can also be filledin advance in a hollow capsule 3 composed of two symmetrical half-capsules 31, 32 butted to facilitate the consumption by the eater. The hollow capsule 3 includes gelatin or vegetable ingredients that can be absorbed by the human body, such as methyl cellulose, hydroxypropyl cellulose, and propyl methyl cellulose.

What is claimed is:

1. An expandable microcapsule, comprising:
a core layer consisting of 300 parts by weight of foaming agent, 250 parts by weight of polymer water-absorbing material and 60 parts by weight of core layer lubricant, wherein the foaming agent has characteristics of reacting with water to generate gas; and
a coating layer covering the surface of the core layer to form a granular structure, wherein the coating layer is consisting of 10% to 40% by weight of edible water-absorbent colloid, 5% to 40% by weight of functional health food additives, 2% to 30% by weight of emulsifier, 4% to 15% by weight of coating layer lubricant and 5% to 40% by weight of film coating composition, and wherein the film coating composition has characteristics of resistance to gastric acid decomposition and being decomposed only in the intestinal tract.

2. The expandable microcapsule according to claim 1, wherein the foaming agent is consisting of 100 to 300 parts by weight of an acidic compound and 130 to 410 parts by weight of a basic compound, wherein the acidic compound is composed of at least one of citric acid or tartaric acid, and the basic compound is composed of at least one of sodium carbonate, sodium bicarbonate, potassium carbonate, and potassium bicarbonate.

3. The expandable microcapsule according to claim 1, wherein the polymer water-absorbing material is sodium polyacrylate, wherein the polymer water-absorbing material is superfinely pulverized to a size of 300 mesh or more, and dried at 40° C. to 80° C. for 5 hours.

4. The expandable microcapsule according to claim 1, wherein the core layer lubricant is consisting of 20 to 60 parts by weight of an internal lubricant and 2 to 6 parts by weight of an external lubricant, wherein the internal lubricant is composed of one or a combination of polyethylene glycol 4000 and polyethylene glycol 6000, and the external lubricant is composed of magnesium stearate, talc, or one or a combination of L-leucine and DL-leucine.

5. The expandable microcapsule according to claim 4, wherein the core layer is made by mixing with a foaming agent, a polymer water-absorbing material and an internal lubricant, then adding 240 parts by weight of a soft material made of anhydrous ethanol, granulated by sieving, and dried at 40° C. to 80° C. to make the water content of the mixture particles below 3%, and then adding an external lubricant and mixing.

6. The expandable microcapsule according to claim 1, wherein the edible water-absorbing colloid is composed of pectin, konjac powder, arabic gum, carrageenan, alginate, agar, guargum, xanthan gum, locust bean gum, gelatin, gellan gum, galactomannan, tragacanth gum, karaya gum, curdlan gum, chitosan, xyloglucan, β-glucan, furcellaran, Ghattigum, tamarin, bacterial gum, propylene glycol alginate, carboxymethyl locust bean gum, low methoxy pectin, mixture of emulsified calcium and pectin, modified microcrystalline cellulose, modified carboxymethyl cellulose (CMC), modified methyl cellulose (MC), modified hydroxypropyl methyl cellulose (HPMC), modified hydroxypropyl cellulose (HPC), or any combination thereof.

7. The expandable microcapsule according to claim 1, wherein the film coating composition is consisting of one of 1% to 25% by weight of film-forming agent, 0.5% to 15% by weight of anti-sticking agent, and 60% to 95% by weight of solvent, and the film-forming agent is composed of shellac methyl cellulose, ethyl cellulose, propyl methyl cellulose, hydroxypropyl methyl cellulose phthalate (HPMCP), pectin, seaweed gum, sodium alginate, hydroxypropyl cellulose, hydroxypropyl methyl cellulose, hydroxyethyl cellulose, polyvinylpyrrolidone, polyethylene glycol, aminoalkyl methacrylate copolymer, maltodextrin and polydextrose or any combination thereof.

8. The expandable microcapsule according to claim 1, wherein the emulsifier is selected from one of lecithin, fatty ester of glycerol, sucrose and sorbitol.

9. The expandable microcapsule according to claim 1, wherein the coating layer lubricant is composed of one of polyethylene glycol 4000, polyethylene glycol 6000, sodium lauryl sulfate, magnesium lauryl sulfate, L-leucine, sodium benzoate, sodium oleate, sodium chloride, sodium acetate, boric acid, magnesium stearate, talc, micronized silica gel, sucrose fatty acid ester, sodium stearyl fumarate, L-leucine and DL-leucine, or any combination thereof.
